(19) United States
(12) Patent Application Publication
(10) Pub. No.: US 2003/0044478 A1
(43) Pub. Date: Mar. 6, 2003

(54) Burnet extract

(76) Inventors: Howard A. Epstein, Cincinnati, OH (US); Christine Hall, Cincinnati, OH (US)

Correspondence Address: Supervisor, Patent Prosecution Services, PIPER MARBURY RUDNICK & WOLFE LLP, 1200 Nineteenth Street, N.W., Washington, DC, 20036-2412, US

(21) Appl. No.: 09/929,122

(22) Filed: Aug. 15, 2001

(51) Int. Cl. A61K 35/78

(52) U.S. Cl. 424/765

(57) ABSTRACT

The present invention relates to burnet extract and to external skin compositions comprising the burnet extract. The invention also relates to a method of preparing the burnet extract.

### BACKGROUND OF THE INVENTION

1. Field of the Invention

The present invention relates to a burnet extract and a process for its extraction. The present invention further relates to compositions comprising the burnet extract.

2. Discussion of the Background

Burnet plants (Sanguisorba officinalis L.) belong to the family of plants known as Rosaceae. The Rosaceae family is a large group containing over 100 genera and about 3,000 species of herbs, shrubs, vines and trees.

Plant extracts selected from various members of the Rosaceae family exhibit excellent hair growth inhibition. For example, hydrolyzed almond, burnet, rose hips and hawthorn all have been shown to inhibit hair growth. By “hydrolyzed almond” is meant a mixture available by the hydrolysis of seeds of almond (Prunus amygdalus Batsch) in the presence of an acid or alkali. The term “burnet” refers to the root and rootstock of burnet (Sanguisorba officinalis L.). “Rose hips” refers to the fruit of a wild rose (Rosa multiflora Thunberg) or a plant analogous thereto. The term “hawthorn” means the terrestrial part of the hawthorn (Craegus oxyacanth L.).

Burnet has bitter and sour tastes and a slightly cold property. Burnet is employed mainly for its astringent action, being used to slow or arrest blood flow. Chinese herbal medicine has used the leaves and roots of the burnet plant as an anodyne, astringent, contraceptive, diuretic, febrifuge, hemostatic, tonic and vulnerary.

Burnet leaves are astringent, refrigerant, styptic and tonic, while the root is anodyne, astringent, diuretic, febrifuge, hemostatic, tonic and vulnerary. Both the leaves and roots of the burnet plant contain active constituents responsible for these properties. The most active of these constituents include (+)-catechin, quercetin, sanguiin-H-2, sanguiin-H-6, tannin and ursolic acid. In addition, some of the actions of Sanguisorba officinalis are a result of scavenging of nitric oxide (NO) and inhibition of NO formation. This activity appears to be mediated by sanguiin-H-6 and tannin.

Burnet may be taken internally or applied externally. Internally, burnet typically acts on the liver, stomach and large intestine channels.

Burnet functions to dispel heat from blood and to stop bleeding. Burnet root is thought to stop blood flow when taken internally (i.e., for peptic ulcers, menstrual problems, diarrhea, hemorrhoids, etc.). Burnet is especially useful in treating bleeding because of damp-heat associated with such problems as blood in the stool, bleeding hemorrhoids, bloody dysenterial disorders and excessive uterine bleeding. Burnet may also be used to control vomiting of blood and nosebleeds.

Burnet also dispels toxins and promotes healing of ulcers and other sores. Burnet may be applied topically for sores, boils, eczema and injuries from scalds and burns, etc. Burnet both reduces the oozing by holding in the fluids and promotes generation of new flesh.

To date, the standard method of preparation of burnet extract is to soak burnet root in an ethyl alcohol and water menstruum for three to five days, after which the menstruum is mixed, ground and filtered.

### SUMMARY OF THE INVENTION

It is an object of the present invention to provide a burnet extract and a method of preparing burnet extract.

Yet another object of the present invention is to provide various applications for burnet extract.

In accordance with one embodiment of the present invention, there is provided a burnet extract and a method of preparing the extract. In accordance with another embodiment of the present invention, the present invention relates to external skin compositions comprising the burnet extract. The burnet extract of the present invention and the external skin compositions incorporating the same are effective in inhibiting hair growth, treating and preventing inflammation of hair follicles (referred to as “folliculitis”), boosting, or increasing, the SPF factor in sunscreens and acting as an antioxidant.

### DETAILED DESCRIPTION OF THE INVENTION

The present invention provides a burnet extract comprising from about 0.25 percent to about 20 percent by weight burnet-derived compounds. Preferably, the burnet extract of the present invention comprises from about 0.5 percent to about 10 percent by weight burnet-derived compounds. More preferably, the burnet extract comprises about 3 percent by weight burnet-derived compounds.

The burnet extract of the present invention is typically a solvent extract, or diluted solution, a concentrate or a dried powder thereof. The extract is preferably obtained by soaking the burnet in an extracting solvent, or menstruum. The extraction may be performed under refrigeration, if desired.

The present invention, therefore, also provides a method of preparing the burnet extract comprising soaking burnet root in a menstruum. The burnet root may be soaked in the menstruum whole, sliced or ground. The root may be soaked for anywhere from about 36 hours to about 7 days. If desired, the soaking of the burnet root may occur under refrigeration. After soaking for the desired time period, the menstruum is then mixed, ground (if not ground before being soaked) and filtered to yield burnet extract.

Suitable examples of the extracting solvent include water, lower alcohols such as ethanol, propanol and butanol, polyols such as propylene glycol, 1,3-butylene glycol and glycerin and mixtures thereof. Preferred extracting solvents include water, lower alcohols and mixtures thereof. A mixture of water and ethanol is most preferred.

Most preferably, the method of preparing burnet extract according to the present invention comprises soaking burnet root in a menstruum of ethanol and water for about three to about seven days. If desired, the soaking of the burnet root may be done under refrigeration. The menstruum is then mixed, ground (if not pre-ground before use) and filtered to yield burnet extract.

Furthermore, the present invention provides a variety of external skin compositions comprising the burnet extract for such applications as inhibiting hair growth, treating and preventing folliculitis, acting as an antioxidant and boosting the SPF factor of sunscreens and of compositions containing sunscreens.

Accordingly, the above-described burnet extract can be incorporated into any of a number of external skin compositions. The burnet extract is included in the external skin composition in an amount from about 0.5 percent to about 20 percent by weight of the composition. Preferably, the burnet extract comprises from about 2 percent to about 7 percent by weight of the external skin composition. More preferably, the burnet extract comprises about 3 percent by weight of the external skin composition.

The external skin compositions of the present invention can be prepared in various forms in any conventional manner. Preferred examples include lotions, suspensions, creams, emulsions, ointments, sticks, solution in an organic solvent, purified water or the like, facial packs and gels. In other words, the external skin composition of the present invention may be in the form of a skin treatment lotion, a facial freshener, a softening lotion, an acne treatment lotion, an after-shave lotion, a sunscreen lotion, a cleansing composition including cleansing creams and the like, a skin cream, an antiperspirant and/or deodorant and a make-up base, but the invention is not limited to these.

In addition to the burnet extract, any component may also be incorporated into the external skin composition insofar as such component does not impair the advantageous effects of the burnet extract solution. Non-limiting examples of such desired components include purified water, alcohols, chelating agents, oil components, humectants, surfactants, emollients, thickening agents, emulsifiers, antiseptics, antioxidants, solvents, drug efficacy ingredients, powders, ultraviolet absorbers, colorants, perfumes and emulsion stabilizers.

Suitable examples of the oil component include liquid paraffin, petrolatum, paraffin wax, squalane, bees wax, carnauba wax, olive oil, mineral oil, lanolin, higher alcohols, fatty acids, synthetic ester oils between a higher alcohol and fatty acid and silicone oil. Suitable examples of the thickening agents include methacrylates and water-soluble polymers such as carboxyvinyl polymer, carboxymethyl cellulose, polyvinyl alcohol, carrageenan and gelatin. Additional suitable thickening agents for cleansing compositions include electrolytes such as sodium chloride and potassium chloride. Suitable examples of antiseptics include urea, methylparaben, ethylparaben and sodium benzoate. Suitable examples of powders include talc, starch, sericite, mica, mica titanium, kaolin, silica, bentonite, vermiculite, zinc powder, titanium oxide, magnesium oxide, zirconium oxide, barium sulfate, red oxide, iron oxide and ultramarine.

Skin conditioning agents are materials that provide a skin conditioning benefit. For example, the skin conditioning agents provide a therapeutic or cosmetic benefit to the skin including, but not limited to, moisturization, emolliency, visual improvement of the skin surface, soothing of the skin and softening of the skin. Suitable skin conditioning agents include, but are not limited to, dimethicone, cyclomethicone, dimethicone copolyol, ester oils, glyceryl esters such as glyceryl dilaurate and fatty acids such as stearic acid.

Humectants act as hydroscopic agents and increase the amount of water held in the outer layer of the skin, which contributes to the softening of the skin surface. Non-limiting examples of suitable humectants are sorbitol, xylitol, glycerin, maltitol, propylene glycol, 1,3-butylene glycol, 1,4-butylene glycol, sodium pyrrolidonecarboxylate, lactic acid, sodium lactate, polyoxypropylene fatty acid ester and polyethylene glycol.

The emulsifiers used in the present invention are selected from the group consisting of anionic, cationic and nonionic surfactants and mixtures thereof. Most anionic surfactants can be broadly described as having a hydrophobic moiety such as a carbon chain including about 8 to about 30 carbon atoms and, particularly, about 12 to about 20 carbon atoms, and as further having a hydrophilic moiety such as sulfate, stearate, sulfonate, palmitate, carbonate, phosphate or carboxylate. Often, the hydrophobic carbon chain is etherfied, such as with ethylene oxide or propylene oxide, to impart a particular physical property, such as increased water solubility or reduced surface tension, to the anionic surfactant.

Non-limiting suitable anionic surfactants include, therefore, compounds in the classes known as alkyl sulfates, alkyl ether sulfates, alkyl ether sulfonates, sulfate esters of alkylphenoxy polyoxyethylene ethanol, alpha-olefin sulfonates, beta-alkoxy alkane sulfonates, alkylaryl sulfonates, alkyl monoglyceride sulfates, alkyl mono glyceride sulfonates, alkyl carbonates, alkyl ether carboxylates, fatty acids, sulfosuccinates, sarcosinates, octoxynol or nonoxynol phosphates, taurates, fatty taurides, fatty acid amide polyoxyethylene sulfates, isethionates or mixtures thereof. Additional anionic surfactants are listed in McCutcheon's Detergents and Emulsifiers, 1993 Annuals, published by McCutcheon Division, MC Publishing Co., Glen Rock, N.J., pp. 263-266; and in the CTFA Cosmetic Ingredient Handbook, J. M. Nikitakis, ed., published by the Cosmetic, Toiletry and Fragrance Association, Inc., Washington, D.C. (1988) pp. 10-13, 42-46 and 87-94, both incorporated herein by reference.

Specific preferred anionic surfactants include, but are not limited to, lauryl sulfates, octyl sulfates, 2-ethylhexyl sulfates, lauramine oxide, decyl sulfates, tridecyl sulfates, cocoates, lauroyl sarcosinates, lauryl sulfosuccinates, linear C₁₀ diphenyl oxide disulfonates, lauryl sulfosuccinates, lauryl ether sulfates (1 and 2 moles ethylene oxide), myristyl sulfates, oleates, palmitates, stearates, tallates, cetyl sulfates and similar anionic surfactants.

Suitable examples of cationic surfactants include, but are not limited to, quaternary ammonium salts such as distearyldimonium chloride; alkyldimethylbenzylammonium salts; alkyltrimethylammonium salts such as stearyltrimethylammonium chloride and lauryltrimethylammonium chloride; dialkyldimethylammonium salts such as distearyldimethylammonium chloride; alkylpyridinium salts such as poly(N,N′-dimethyl-3,5-methylenepiperidinium) chloride and cetylpyridinium chloride; polyoxyethylene alkylamines; alkylamine salts; and polyamine fatty acid derivatives.

The nonionic surfactants may be straight-chain or branched. Non-limiting examples of suitable nonionic surfactants that may be used in the present invention are polyoxyethylene alkyl ethers such as polyoxyethylene lauryl ether, polyoxyethylene oleyl ether, polyoxyethylene stearyl ether, polyoxyethylene behenyl ether, polyoxyethylene 2-octyldodecyl ether and polyoxyethylene cholestanol ether; polyoxyethylene fatty acid esters such as polyoxyethylene monooleate, polyoxyethylene distearates, polyoxyethylenemonodioleates and polyoxyethylene ethylene glycol distearates; polyoxyethylene sorbitan fatty acid esters such as polyoxyethylene sorbitan monooleate, polyoxyethylene sorbitan monostearate, polyoxyethylene sorbitan monoolate and polyoxyethylene sorbitan tetraoleate; glycerin fatty acid esters; polyglycerin fatty acid esters; polyoxyethylene glycerin fatty acid esters such as polyoxyethylene glycerin monostearate, polyoxyethylene glycerinmonoisostearate and polyoxyethylene glycerin triisostearate; polyoxyethylene castor oil or polyoxyethylene hydrogenated castor oil derivatives such as polyoxyethylene castor oil, polyoxyethylene hydrogenated castor oil, polyoxyethylene hydrogenated castor oil monoisostearate, polyoxyethylene hydrogenated castor oil triisostearate, monopyroglutamate and polyoxyethylene hydrogenated castor oil maleate; polyoxyethylene sorbitol fatty acid esters such as polyoxyethylene sorbitol monostearate, polyoxyethylene sorbitol monolaurate, polyoxyethylene sorbitol monooleate and polyoxyethylene sorbitol pentaoleate; alkanol amides such as coconut oil fatty acid diethanolamide, lauric acid monoethanolamide and fatty acid isopropanolamide; polyoxyethylene alkylamines; and polyoxyethylene fatty acid amides.

Numerous other nonionic surfactants are disclosed in McCutcheon's Detergents and Emulsifiers, 1993 Annuals, published by McCutcheon Division, MC Publishing Co., Glen Rock, N.J., pp. 1-246 and 266-272; in the CTFA International Cosmetic Ingredient Dictionary, Fourth Ed., published by the Cosmetic, Toiletry and Fragrance Association, Inc., Washington, D.C. (1991), pp. 1-651; and in the CTFA Cosmetic Ingredient Handbook, J. M. Nikitakis, ed., published by the Cosmetic, Toiletry and Fragrance Association, Inc., Washington, D.C. (1988) pp. 86-94, each incorporated herein by reference.

In addition, fatty alcohols (typically monohydric alcohols) may be used in the compositions to help to stabilize the emulsion. Preferred fatty alcohols include cetearyl alcohol, cetyl alcohol and stearyl alcohol. Other emulsion stabilizers used in the compositions of the invention include acrylates/C₁₀₋₃₀ alkyl acrylate crosspolymer, nonionic and anionic polymer systems such as, for example, carbomers, and mixtures thereof.

Additional ingredients include, but are not limited to, fragrances, pH adjusters to obtain a pH that matches the natural pH of the skin, sunscreens such the p-aminobenzoic acid derivatives or any other sunscreen having the ability to block the sun's rays and anti-acne agents such as salicylic acid and their derivatives or benzoyl peroxide to fight unwanted and unsightly acne blemishes. The external skin compositions may also contain other conventional additives commonly employed in cosmetic emulsions. For example, the inventive compositions may contain one or more colorants. In particular, a whitening pigment such as titanium oxide may be added to provide an aesthetically pleasing color to the external skin compositions. The compositions may also contain fragrances which are non-irritating to the skin and soothing to the olfactory system.

Preservatives are routinely added to cosmetic emulsions to protect against the growth of microorganisms. Cosmetic chemists are familiar with appropriate preservatives and will choose them to satisfy the particular composition. The preservatives should be selected according to the use of the compositions and any possible incompatibilities between the preservatives and the other ingredients in the compositions. Non-limiting examples of commonly used preservatives include methylparaben, propylparaben, polyparaben, DMDM hydantoin and phenoxyethanol. Typically, the balance of the external skin compositions is water.

Having generally described this invention, a further understanding can be obtained by reference to specific examples. The examples are provided herein for purposes of illustration only and are not intended to be limiting unless otherwise specified.

### EXAMPLES

### Extract Preparation Example

Burnet root obtained from burnet plants (Sanguisorba officinalis L.) is sliced. The burnet root is added to a menstruum of about 20 parts to about 65 parts ethanol and about 50 parts to about 95 parts water in an amount such the burnet root is about 8 parts to about 15 parts of the menstruum. The burnet root is soaked in the menstruum for about seven days. The menstruum is then mixed, ground and filtered to yield burnet extract solution. The resulting burnet extract contains about 3 percent burnet-derived compounds.

### Composition Examples

According to the formulation described below, a skin moisturizing composition incorporating the burnet extract of the present invention was prepared in a conventional manner.

| Ingredient | Use Level (% W/W) |
|---|---|
| Water | q.s. to 100% |
| Methylparaben | 0.200 |
| Sodium hydroxide | 0.080 |
| Cetearyl alcohol | 3.300 |
| Ceteareth-20 | 1.350 |
| Glyceryl dilaurate | 0.500 |
| Glycerin | 6.000 |
| Stearic acid | 0.500 |
| Mineral oil | 1.500 |
| Petrolatum | 2.000 |
| Propylparaben | 0.100 |
| Aluminum starch octenylsuccinate | 1.000 |
| Dimethicone | 0.500 |
| Cyclomethicone and Dimethicone | 1.000 |
| Acrylates/C₁₀₋₃₀ alkyl acrylate crosspolymer | 0.100 |
| DMDM hydantoin | 0.400 |
| Fragrance | 0.100 |
| Burnet extract (water and ethanol) | 3.000 |

In a twelve-week study of the efficacy of burnet extract to inhibit hair growth, ten volunteers applied the above-described composition to one of their legs. To their other leg, they applied a control composition that did not contain burnet extract. Each composition was applied to the respective legs twice a day. After twelve weeks, seven of the ten (70%) of the volunteers reported that they did not have to shave the leg to which they applied the composition containing burnet extract as often as they did the leg to which they applied the control composition.

In a separate study, over 100 volunteers were given the above-described composition to use as a moisturizer for their legs for an eight-week period. At the four-week check point, a majority of the panelists were able to recognize the benefits as indicated below in Table I.

| Benefit | % Agree/Strongly Agree |
|---|---|
| My hair is softer | 72.9 |
| My hair is easier to shave | 65.4 |
| Helps me shave less often | 59.4 |
| My hair/stubble is less noticeable | 60.7 |
| Keeps my legs smoother longer | 85.8 |
| Delays the appearance of unsightly | 59.8 |
| stubble | |
| My hair is softer and finer | 66.4 |

Both of these studies indicate that the burnet extract of the present invention exhibits excellent hair growth inhibitory action.

According to the formulations described below, several additional skin moisturizing compositions incorporating the burnet extract of the present invention were prepared in a conventional manner.

Skin Moisturizing Composition #1

| Ingredient | Use Level (% W/W) |
|---|---|
| Water | q.s. to 100% |
| Glycerin | 12.00 |
| Distearyldimonium chloride | 5.00 |
| Petrolatum | 4.00 |
| Isopropyl palmitate | 3.00 |
| Burnet extract (water and ethanol) | 3.00 |
| Cetyl alcohol | 2.50 |
| Dimethicone | 0.50 |
| Methylparaben | 0.20 |
| Propylparaben | 0.10 |
| Fragrance | 0.10 |

Skin Moisturizing Composition #2

| Ingredient | Use Level (% W/W) |
|---|---|
| Water | q.s. to 100% |
| Glycerin | 4.50 |
| SD alcohol 40B | 4.50 |
| Burnet extract (water and ethanol) | 3.00 |
| Cetyl esters wax | 2.20 |
| Glyceryl dilaurate | 1.85 |
| Cetyl alcohol | 1.70 |
| Cetearyl alcohol | 1.60 |
| Ceteareth-20 | 0.62 |
| Lanolin oil | 0.55 |
| Dimethicone | 0.50 |
| DMDM hydantoin | 0.40 |
| Methylparaben | 0.20 |
| Carbomer | 0.15 |
| Fragrance | 0.10 |
| Sodium hydroxide | 0.06 |

Skin Moisturizing Composition #3

| Ingredient | Use Level (% W/W) |
|---|---|
| Water | q.s. to 100% |
| Glycerin | 6.00 |
| Cetearyl alcohol | 2.20 |
| Burnet extract (water and ethanol) | 3.00 |
| Mineral oil | 2.50 |
| Aluminum starch | 2.00 |
| Petrolatum | 2.00 |
| Palm glyceride | 1.50 |
| Ceteareth-20 | 1.30 |
| Dimethicone | 1.00 |
| C₁₂₋₁₅ alcohol benzoate | 1.00 |
| Distearyldimonium chloride | 0.90 |
| Cetyl alcohol | 0.50 |
| DMDM hydantoin | 0.40 |
| Methylparaben | 0.20 |
| Polyquaternium 10 | 0.10 |
| Propylparaben | 0.10 |
| Tetrasodium EDTA | 0.10 |
| Fragrance | 0.10 |

Skin Moisturizing Composition #4

| Ingredient | Use Level (% W/W) |
|---|---|
| Water | q.s. to 100% |
| Glycerin | 4.00 |
| Cetearyl alcohol | 3.80 |
| Burnet extract (water and ethanol) | 3.00 |
| Palm glyceride | 1.50 |
| Ceteareth-20 | 1.30 |
| Cetyl alcohol | 1.00 |
| C₁₂₋₁₅ alkyl benzoate | 1.00 |
| Glyceryl dilaurate | 1.00 |
| Cyclomethicone and Dimethicone | 0.50 |
| DMDM hydantoin | 0.40 |
| Stearic acid | 0.25 |
| Methylparaben | 0.20 |
| Acrylates/C₁₀₋₃₀ alkyl acrylate crosspolymer | 0.10 |
| Propylparaben | 0.10 |
| Fragrance | 0.10 |
| Sodium hydroxide | 0.05 |

Skin Moisturizing Composition #5

| Ingredient | Use Level (% W/W) |
|---|---|
| Water | q.s. to 100% |
| Glycerin | 8.00 |
| Cetearyl alcohol | 3.30 |
| Burnet extract (water and ethanol) | 3.00 |
| Petrolatum | 2.00 |
| Cyclomethicone and Dimethicone | 2.00 |
| Mineral oil | 1.50 |
| Ceteareth-20 | 1.35 |
| Aluminum starch octenylsuccinate | 1.00 |
| Glyceryl dilaurate | 0.50 |
| Stearic acid | 0.50 |
| DMDM hydantoin | 0.40 |
| Methylparaben | 0.20 |
| Acrylates/C₁₀₋₃₀ alkyl acrylate crosspolymer | 0.12 |
| Propylparaben | 0.10 |
| Fragrance | 0.10 |
| Sodium hydroxide | 0.50 |

Skin Moisturizing Composition #6

| Ingredient | Use Level (% W/W) |
|---|---|
| Water | q.s. to 100% |
| Glycerin | 8.00 |
| Burnet extract (water and ethanol) | 3.00 |
| Dimethicone | 2.50 |
| Paragon III (Preservative system: DMDM | 1.20 |
| hydantoin, methylparaben and propylparaben) | |
| Isododecane | 1.00 |
| Acrylates/C₁₀₋₃₀ alkyl acrylate crosspolymer | 0.70 |
| PEG-20 sorbitan isostearate | 0.50 |
| Tetrasodium EDTA | 0.30 |
| Sodium hydroxide | 0.25 |
| Fragrance | 0.10 |

Having now fully described the invention, it will be apparent to one of ordinary skill in the art that changes and modifications can be made thereto without departing from the spirit or scope of the invention as set forth herein. Unless such changes and modifications depart from the scope of the invention, they should be construed as being included therein. It is intended, therefore, that the foregoing detailed description be understood from the following claims, including all equivalents, which are intended to define the scope of the invention.

What is claimed is:

1. A burnet extract comprising from about 0.25 percent to about 20 percent by weight burnet-derived compounds.

2. The solution of claim 1, wherein the burnet extract comprises from about 0.5 percent to about 10 percent by weight burnet-derived compounds.

3. The solution of claim 1, wherein the burnet extract comprises about 3 percent by weight burnet-derived compounds.

4. A method of inhibiting hair growth comprising administering the burnet extract of claim 1.

5. The method of claim 4, wherein the burnet extract is administered to the region of the skin where hair growth inhibition is desired.

6. A method of treating and preventing folliculitis comprising administering the burnet extract of claim 1.

7. A method of increasing the SPF factor in sunscreens, wherein the method comprises administering a sunscreen comprising the burnet extract of claim 1.

8. A method of increasing the SPF factor in compositions comprising sunscreens, wherein the method comprises administering a composition comprising a sunscreen and the burnet extract of claim 1.

9. A method of preparing a burnet extract, comprising:
soaking burnet root in a menstruum; and
mixing and filtering the menstruum to obtain the burnet extract.

10. The method of claim 9, wherein the menstruum comprises ethanol and water.

11. The method of claim 9, wherein the burnet root is soaked in the menstruum for about 36 hours to about 7 days.

12. The method of claim 9, wherein the burnet root is soaked in the menstruum for about 3 days to about 7 days.

13. The method of claim 9, wherein the soaking of the burnet root in the menstruum is done under refrigeration.

14. The method of claim 9, wherein whole or sliced burnet root is soaked in the menstruum and wherein the menstruum is ground before filtering.

15. The method of claim 9, wherein the burnet root is ground before soaking the burnet root in the menstruum.

16. An external skin composition comprising the burnet extract of claim 1.

17. The external skin composition of claim 16, wherein the composition is selected from the group consisting of a skin treatment lotion, a facial freshener, a moisturizing lotion, an acne treatment lotion, an after-shave lotion, a sunscreen lotion, a cleansing cream, a skin cream, an antiperspirant and/or deodorant and a make-up base.

18. A moisturizing lotion comprising the burnet extract of claim 1.

19. The moisturizing lotion of claim 18, further comprising:
at least one oil component;
at least one skin conditioning agent;
at least one humectant;
at least one emulsifier; and
at least one emulsion stabilizer.

20. A method of inhibiting hair growth, comprising applying a moisturizing lotion comprising the burnet extract of claim 1 to the skin.
